(19) United States
(12) Patent Application Publication
(10) Pub. No.: US 2024/0062910 A1
(43) Pub. Date: Feb. 22, 2024

(54) METHOD FOR PREDICTING RISK OF DEPRESSION RELAPSE OR ONSET

(71) Applicant: Feel Therapeutics Inc., San Francisco, CA (US)

(72) Inventors: Georgios Eleftheriou, San Francisco, CA (US); Panagiotis Fatouros, Athens (GR); Charalampos Tsirmpas, San Francisco, CA (US)

(21) Appl. No.: 18/235,510

(22) Filed: Aug. 18, 2023

Related U.S. Application Data
(60) Provisional application No. 63/398,983, filed on Aug. 18, 2022.

(51) Int. Cl. G16H 50/30; G16H 50/20; G16H 40/67; G16H 20/10; G06F 21/60; A61B 5/16; A61B 5/00

(52) U.S. Cl. CPC G16H 50/30; G16H 50/20; G16H 40/67; G16H 20/10; G06F 21/602; A61B 5/165; A61B 5/74; A61B 5/4803; A61B 5/4848; A61B 5/4842; A61B 5/6801; G06F 40/35

(57) ABSTRACT

A method includes, during a first time, for each user in a population: accessing a clinical assessment for depression in the user; accessing a set of biosignal data of the user; deriving correlations between the set of biosignal data and the clinical assessment for depression; and compiling the correlations into a depression model configured to predict risk of future depression diagnosis. The method further includes, during a second time: accessing a series of biosignal data collected by a device worn by a first user; accessing a target duration; calculating a risk of presentation of depression symptoms by the first user within the target duration based on the series of biosignal data; and in response to the risk exceeding a threshold, serving a notification to a care provider associated with the first user, the notification indicating the risk and including a prompt to investigate the first user for treatment.

### CROSS-REFERENCE TO RELATED APPLICATIONS

This Application claims the benefit of U.S. Provisional Application No. 63/398,983, filed on 18 Aug. 2022, which is incorporated in its entirety by this reference.

This Application is related to U.S. patent application Ser. No. 16/460,105, filed on 2 Jul. 2019, and to U.S. patent application Ser. No. 18/126,100, filed on 24 Mar. 2023, each of which is incorporated in its entirety by this reference.

### TECHNICAL FIELD

This invention relates generally to the field of biosensors and more specifically to a new and useful method for detecting and monitoring clinical depression and anxiety biomarkers in the field of biosensors.

### BRIEF DESCRIPTION OF THE FIGURES

FIG. 1 is a flowchart representation of a method;

FIG. 2 is a flowchart representation of a variation of the method;

FIG. 3 is a schematic representation of a variation of the method;

FIG. 4 is a flowchart representation of a variation of the method;

FIG. 5 is a flowchart representation of a variation of the method; and

FIG. 6 is a flowchart representation of a variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

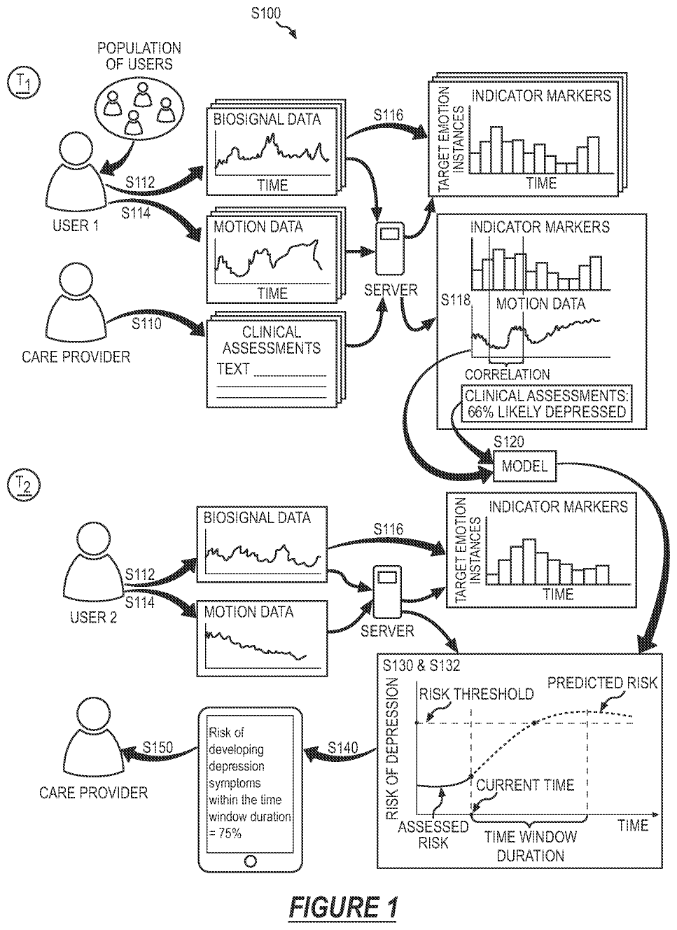

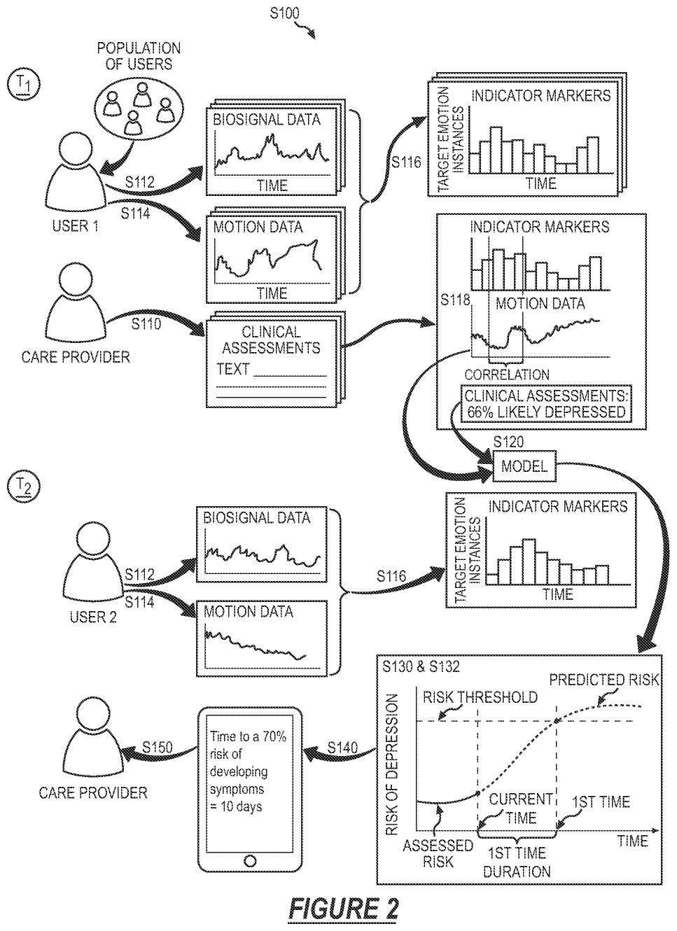

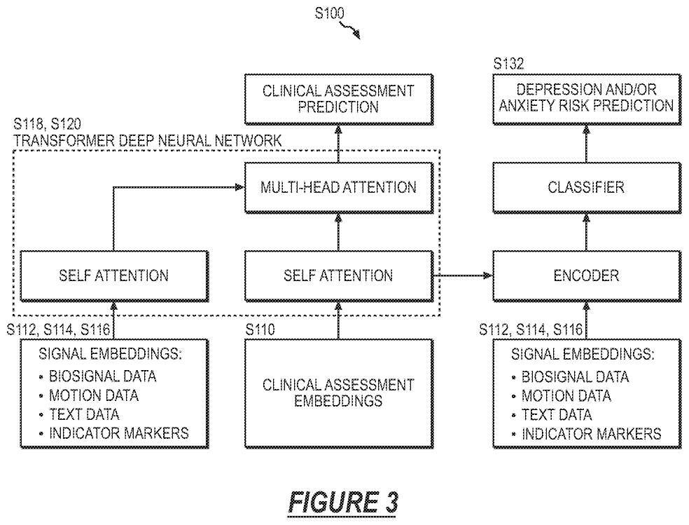

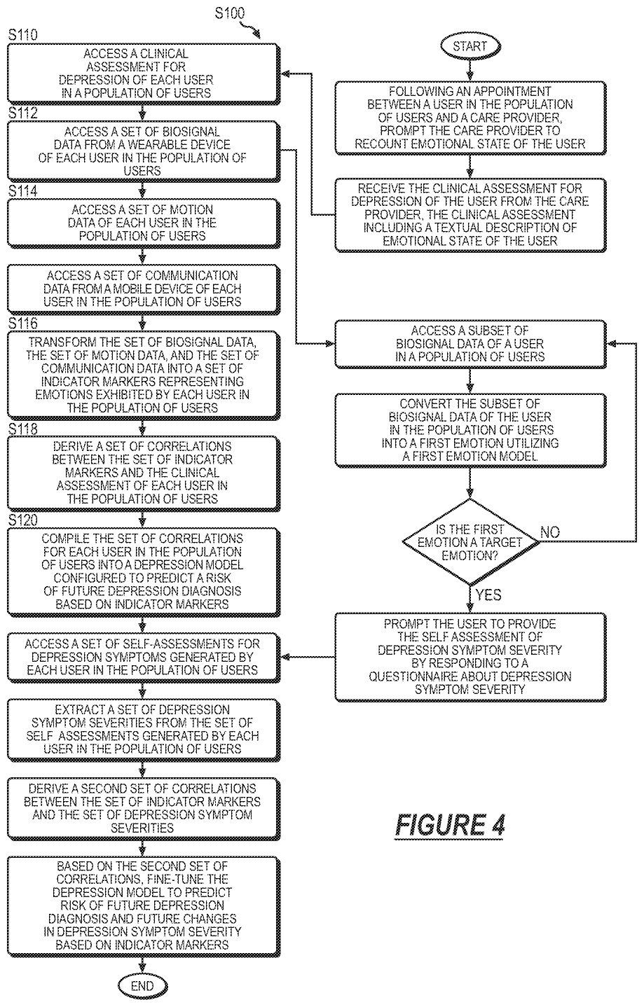

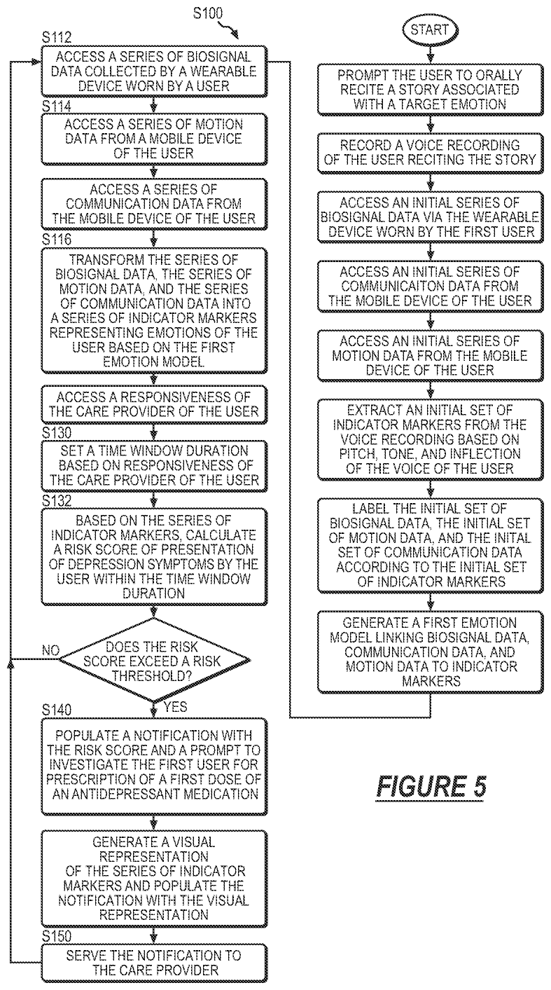

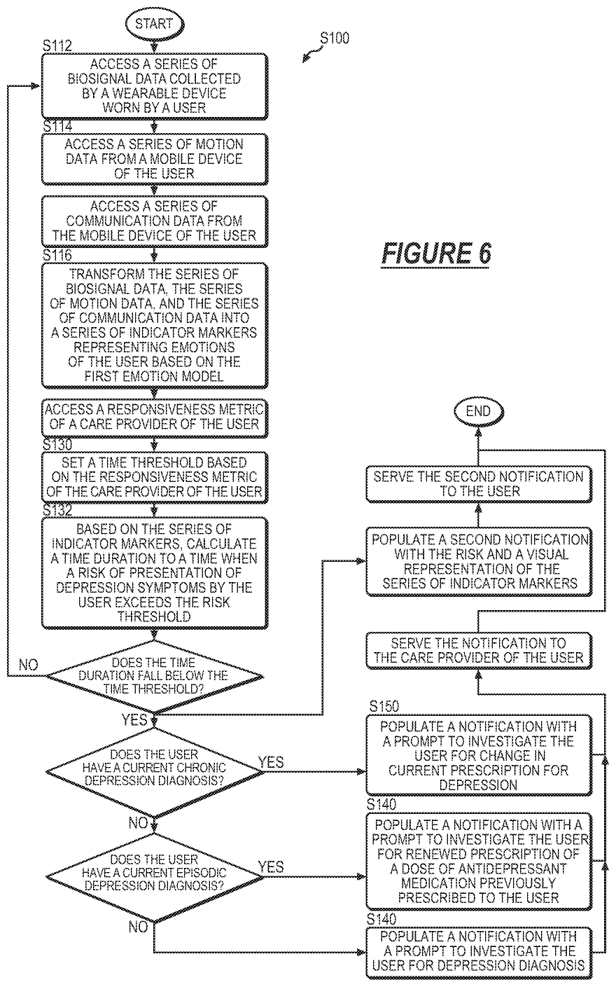

As shown in FIGS. 1-6, a method S100 includes, during a first time period, for each user in a population of users: accessing a clinical assessment for depression in the user in Block S110; accessing a set of biosignal data, of the user, preceding the clinical assessment for depression in Block S112; accessing a first set of motion data, of the user, preceding the clinical assessment for depression in Block S114; transforming the set of biosignal data into a set of psychophysiological markers, the first set of psychophysiological markers representing emotions exhibited by the user in Block S116; and deriving a set of correlations between the set of psychophysiological markers and the clinical assessment for depression and the first set of motion data and the clinical assessment for depression in Block S118. The method S100 also includes compiling sets of correlations, derived for the population of users, into a depression model configured to predict risk of future depression diagnosis based on historical psychophysiological markers and historical motion data in Block S120.

The method S100 further includes, during a second time period: accessing a series of biosignal data collected by a wearable device worn by a first user in Block S112; accessing a series of motion data of the first user in Block S114; transforming the series of biosignal data into a series of psychophysiological markers of the first user in Block S116; accessing a time risk threshold in Block S130; prior to presentation of depression symptoms by the first user, calculating a first risk score representing presentation of depression symptoms by the first user within the target time window based on the series of psychophysiological markers, the series of motion data, and the depression model in Block S132; in response to the first risk score exceeding a threshold risk, populating a notification with the first risk score and a prompt to investigate the first user for prescription of a first dose of an pharmacological medication in Block. S140; and serving the notification to a care provider associated with the first user in Block S150.

1.1 Variation: Risk of Depression Recurrence within Time Window

In one variation, the method S100 includes, during a first time period, for each user in a population of users: accessing a clinical assessment for depression in the user in Block S110; accessing a set of biosignal data, of the user, preceding the clinical assessment for depression in Block S112; and accessing a first set of motion data, of the user, preceding the clinical assessment for depression in Block S114. The method S100 further includes, during the first time period: deriving a set of correlations between the set of biosignal data and the clinical assessment for depression and between the first set of motion data and the clinical assessment for depression in Block S118; and compiling sets of correlations, derived for the population of users, into a depression model configured to predict risk of future depression diagnosis based on historical biosignal data and historical motion data in Block S120.

In this variation, the method S100 further includes, during a second time period: accessing a series of biosignal data collected by a wearable device worn by a first user in Block S112; accessing a series of motion data collected by a mobile device of the first user in Block S114; accessing a target time window in Block S130; and, prior to presentation of the set of depression symptoms by the first user, calculating a first risk score representing presentation of the set of depression symptoms by the first user within the target time window based on the series of biosignal data and the series of motion data in Block S132. The method S100 also includes: in response to the first risk score exceeding a threshold risk, populating a notification with the first risk score and a prompt to investigate the first user for prescription of a first dose of an antidepressant medication in Block S140; and serving the notification to a care provider associated with the first user in Block S150.

### 1.2 Variation: Time Duration to Threshold Risk

In another variation, the method S100 includes, during a first time period, for each user in a population of users: accessing a clinical assessment for depression in the user in Block S110; accessing a set of biosignal data, of the user, preceding the clinical assessment for depression in Block S112; accessing a first set of motion data, of the user, preceding the clinical assessment for depression in Block S114; and transforming the set of biosignal data and the first set of motion data into a set of psychophysiological markers of the user in Block S116. The method S100 further includes, during the first time period: deriving a set of correlations between the set of psychophysiological markers and the clinical assessment for depression in Block S118; and compiling sets of correlations, derived for the population of users, into a depression model configured to predict risk of future depression diagnosis based on historical psychophysiological markers in Block S120.

In this variation, the method S100 also includes, during a second time period: accessing a series of biosignal data collected by a wearable device worn by a first user in Block S112; accessing a series of motion data of the first user in Block S114; transforming the series of biosignal data and the series of motion data into a series of psychophysiological markers in Block S116; accessing a risk threshold in Block S130; and, prior to presentation of a set of depression symptoms by the first user, calculating a first time associated with a risk of presentation of the set of depression symptoms by the first user exceeding the risk threshold based on the series of psychophysiological markers and the depression model in Block S132 and calculating a first time duration between the first time and a current time in Block S134. The method S100 further includes: in response to the first time duration falling below a threshold duration, populating a notification with the first time duration and a prompt to investigate the first user for prescription of an intervention in Block S140; and serving the notification to a care provider associated with the first user in Block S150.

### 2. Applications

Generally, Blocks of the method S100 can be executed by a companion application executing on a mobile device in cooperation with a wearable device worn by a user and a remote computer system (hereinafter the “computer system”) to assess a risk of recurrence of depression symptoms within a user (e.g., a patient) within a target time window—based on physiological biosignal data (e.g., heart-rate, heart-rate variability, skin temperature, skin moisture, electrodermal activity, etc.), motion data (e.g., data collected by the mobile device of the user, geolocation data, acceleration data, Bluetooth data), and/or communication data (e.g., data collected by the mobile device of the user, voice, video, text, user behavioral data) of the user—prior to the user exhibiting depression symptoms discernible to a medical care provider (e.g., medical clinic staff, medical doctor, nurse, psychiatrist, therapist) and/or to the user herself.

More specifically, the computer system can: retrieve timeseries (e.g., set, series) of biosignal, motion, location, and/or social interaction data, etc. of a population of users; retrieve clinical depression diagnoses of these users and/or depression self-assessments of these users; and compile these user population data into a model configured to predict a) a risk of depression relapse by a user within a target time window (e.g., controlled by a maximum time needed for a clinical depression review, pharmacological medication prescription, and pharmacological medication administration to a user) and/or b) a time duration until risk of depression relapse by a user will exceed a threshold risk (e.g., controlled by pharmacological medication administration procedures). The computer system can then interface with a companion application and a wearable device affiliated with a particular user: to collect biosignal, motion, location, and/or social interaction data, etc. of a particular user; to inject these data into the model to predict a risk of depression relapse by the particular user within a target time window; and to automatically prompt a care provider (e.g., a psychologist, a clinician) to investigate or consider the user for prescription or administration of a pharmacological medication if this predicted risk exceeds a threshold risk. Alternatively, the computer system can: inject these data of the particular user into the model to predict a time duration until risk of depression relapse by the particular user exceeds a threshold risk; and to automatically prompt the care provider to investigate or consider the user for prescription or administration of the pharmacological medication if this predicted time duration is less than a threshold duration.

Therefore, the computer system can: receive user data (e.g., biosignal, motion, location, and/or social interaction data) and corresponding depression diagnoses from a large sample of users (e.g., population of users) collected over a time period; derive correlations between the user data collected earlier within the time period and the corresponding depression diagnoses collected later within the time period for each user in the sample of users; and compile these correlations into a model. The computer system can then utilize the model to correlate new data (e.g., data collected during a subsequent time period) for a particular user with a) risk of depression occurrence (i.e., risk of depression relapse or onset by a user within a target time window) or b) time to depression occurrence (i.e., time duration until risk of depression relapse or onset of a user will exceed a threshold risk) prior to the particular user presenting with human-discernible depression symptoms (i.e., before even a professional or clinical care provider could discern relapse or onset with sufficient confidence to take action).

The computer system can then prompt the care provider to take action (e.g., arrange an appointment with the user, prescribe therapy, prescribe pharmacological medication to the user, administer pharmacological medication to the user) if the risk of depression occurrence is high (e.g., exceeding a risk threshold) or if time duration to depression occurrence or onset is low (e.g., falling below a time threshold) to ensure that the user: receives medical care before experiencing discernible depression symptoms; and receives medical care before depression symptom severity reduces the user's willingness to seek medical attention. In addition, by prompting the care provider to take action (e.g., arrange an appointment with the user, prescribe therapy, prescribe pharmacological medication to the user, administer pharmacological medication to the user), the computer system can enable the care provider to: avoid guessing when the user may need an intervention (e.g., therapy, pharmacological medication, etc.) administered; avoid prematurely administering intervention to the user; avoid erroneously administering additional pharmacological medication (e.g., antidepressant medication) to the user when the additional pharmacological medication does not benefit the user and/or may harm the user (e.g., due to toxicity, side effects, and/or allergies); avoid failing to act when a timely intervention would have positive effect on the user in long term; avoid failing to act when a timely intervention would enable the user to avoid developing depression symptoms.

Therefore, by predicting risk of depression occurrence of a user within a target time window, the computer system can enable the care provider to predict depression occurrence of the user significantly in advance of the predicted depression occurrence. For example, the computer system can predict the risk of depression occurrence (e.g., relapse or onset) weeks or months in advance of the possible occurrence event. Accordingly, the computer system can enable the care provider to respond to the prediction by administering the intervention (e.g., pharmacological medication, therapy) to the user before the user experiences depression symptoms. For example, by predicting the risk of depression occurrence in advance (e.g., weeks or months before the depression occurrence may occur), the computer system can enable a user having poor access to medical care (e.g., long wait times for medical appointments, poor access to pharmacy/medications) to receive the pharmacological medication or medical appointment with the care provider before the depression relapse or onset occurs. In another example, by predicting the risk of depression occurrence in advance (e.g., weeks or months before the depression relapse or onset may occur), the computer system can enable a care provider to administer the pharmacological medication to the user such that the pharmacological medication takes effect prior to the depression occurrence in the user and peak effectiveness of the pharmacological medication corresponds to the predicted depression occurrence event.

### 2.1 Generating the Model

In one implementation, during a first time period, the computer system can prompt a care provider (e.g., coach, psychologist, psychiatrist) of a user in a population of users to complete a set of clinical evaluations configured to evaluate the state (e.g., mental, emotional, cognitive state, emotional awareness, appearance, depression, anxiety, compliance of the user) of the user, which may include indicators of depression relapse or onset. In addition, during the first time period, the computer system can prompt the user in the population of users to complete a set of self-evaluations configured to evaluate symptom severity of the set of depression symptoms of the user. In addition, during the first time period, the computer system can access a set of motion data and a set of communication data stored on the mobile device of the user in the population of users, and upload these data to the computer system. Simultaneously, the wearable device worn by the user in the population of users can upload a set of physiological biosignal data to the computer system. Therefore, the computer system can access the set of clinical evaluations, the set of self-assessments, the set of motion data, the set of communication data, and the set of physiological biosignal data of each user in the population of users.

Then, the computer system can train a depression model (e.g., transformer deep neural network) to identify correlations (e.g., patterns) between user data—including the set of physiological biosignal data, the set of motion data, and the set of communication data—and the set of clinical evaluations. Therefore, the computer system can train the depression model to identify patterns in the user data that are correlated with clinical evaluations, which are indicative of the severity and quantity of depression symptoms exhibited by the user and observed by the care provider.

In one implementation, the computer system can further tune the depression model (e.g., by adjusting parameters of the model) to predict the self-evaluations (e.g., responses to PHQ-9, GAD-7, HDRS, etc.) of a user, which reflect severity of the set of depression symptoms of the user. For example, the computer system can fine-tune the depression model by executing the depression model to predict a set of masked (but known) self-evaluations based on the set of physiological biosignal data, the set of motion data, and the set of communication data of the user. Then, if the predicted self-evaluations in the set of self-evaluations do not match the known self-evaluations, depression model parameters can be adjusted to yield a match between the predicted self-evaluations and the known self-evaluations on subsequent iterations of the fine-tuning process. Accordingly, the depression model is trained to predict changes in severity of the set of depression symptoms of the user.

Therefore, the computer system can train the model to: predict a clinical evaluation for a user, the clinical evaluation indicative of the severity and quantity of depression symptoms exhibited by the user and observed by the care provider; predict anticipated changes in severity of the set of depression symptoms of the user; and, based on the severity, quantity, and the anticipated changes in severity of the set of depression symptoms of the user, calculate a risk of depression relapse or onset in the user within the target time window.

### 2.2 User Onboarding and Risk Prediction

In one implementation, the computer system can onboard the user: diagnosed with a clinical depression disorder (or another complex multi-symptom disorder, such as generalized anxiety) by a medical care provider (hereinafter, “care provider”); and receiving medical care from the care provider to manage her clinical depression disorder. For example, the user may be currently (or previously) prescribed an pharmacological medication (e.g., antidepressant medication) by the care provider. Additionally, or alternatively, the computer system can onboard a user exhibiting non-clinically-significant depressive or anxiety symptoms but for whom potential symptom worsening may require a diagnosis by the care provider.

In this implementation, the wearable device can offload a series of physiological biosignal data to the computer system, such as via the mobile device in real-time or in intermittent data packets. In addition, the companion application can access a series of motion data and a series of communication data on the mobile device and upload these data to the computer system. The computer system can then access a depression model (e.g., transformer deep neural network) trained to predict the risk of depression occurrence (i.e., relapse or onset) based on the series of physiological biosignal data, the series of motion data, and the series of communication data. The remote computer system can therefore leverage the concurrent series of biosignal data, motion data, and communication data, and the depression model to interpret a trajectory of a mental state of the user, and therefore, interpret the risk of the user exhibiting the set of depression symptoms within the target time window.

Furthermore, in response to the risk of the user exhibiting a set of depression symptoms within the target time window exceeding a threshold risk (or falling within a target range of risk levels), the computer system can: generate a notification for the medical provider, the notification indicating the risk and prompting the medical provider to investigate (e.g., set a medical appointment with, review biosignal data of) the user for prescription of a first dose of an pharmacological medication; and serve the notification to a care provider.

In one example, in response to receiving the notification, the care provider may: view the series of physiological biosignal data, the series of motion data, and a series of communication data of the user; based on these data, confirm the risk of depression occurrence in the user within the target time window; review prescription history and/or medical record of the user; and re-prescribe a course of antidepression mediation (e.g., previously-prescribed medication) to the user to prevent the user from developing symptoms of depression within the target time window.

Therefore, the computer system can enable the care provider to: remotely monitor the mental health of the user while avoiding frequent medical appointments between the user and the care provider; assess the risk of the user developing the set of depression symptoms within a given time period; review the data supporting the risk of the depression occurrence in the user occurring within the given time period; and, based on these data, prescribe treatment to the user before the user experiences the set of symptoms associated with depression occurrence. Accordingly, the computer system can enable the user to avoid experiencing a sudden onset of the set of depression symptoms and thereby prevent the user from seeking emergency medical treatment for these symptoms.

In another example, in response to receiving the notification indicating the risk of depression relapse or onset in the user within the target time window exceeding the threshold risk, the care provider may: arrange a medical appointment (e.g., phone call, check-in) with the user to observe the mental state of the user; and confirm the risk of depression relapse or onset in the user within the target time window. The care provider may further: review prescription history and medical records of the user; and, in response to identifying that the user is currently prescribed a first dose of the pharmacological medication, adjust the treatment plan of the user (e.g., by increasing/decreasing the dose of antidepression mediation currently prescribed to the user or prescribing a dose of a second pharmacological medication to the user) to prevent the user from developing the set of symptoms of depression within the target time window.

Therefore, the computer system enables the user—who may be experiencing symptoms of depression including fatigue, lack of concentration, and therefore, may find it difficult to initiate contact with the care provider—to report to the care provider an account of the current mental state to the user. Additionally, the computer system enables the care provider to: monitor response of the user to the currently prescribed pharmacological medication; adjust the dose of the currently prescribed pharmacological medication in response to the user not responding to the dose; and prioritize arranging an appointment with a first user who is at a greater risk of depression relapse (or onset) than with a second user who is at a lesser risk of depression relapse (or onset) within the target time window. Accordingly, the computer system can enable the medical provider to direct medical resources to patients who are at greatest risk of depression relapse (or onset).

In one implementation, in response to the risk of depression relapse (or onset) by the user within the target time window exceeding the threshold risk level, the computer system can serve the notification indicating the risk of depression relapse to the user. In response to receiving the notification, the user may: contact the care provider; arrange an appointment with a therapist; and/or learn to recognize symptoms associated with an increased risk of depression relapse (or onset).

Therefore, the computer system can enable the user to arrange an appointment with the care provider before the user begins to exhibit symptoms associated with depression relapse (or onset) and/or before the symptoms associated with depression relapse begin interfering with the ability of the user to initiate contacting the care provider. Initially, the user may have difficulty discerning a combination of symptoms that can be attributed to depression relapse (or onset) from a second combination of symptoms that can be attributed to daily mood changes of the user. Accordingly, the computer system can enable the user to recognize symptoms associated with an increased risk of depression relapse (or onset), thereby enabling the user to notify the care provider as soon as the symptoms appear.

### 2.2 Predicting a Time Duration to a Risk Threshold

In one variation, based on user data and the model, the computer system can predict a time duration until risk of depression relapse or onset by a user will exceed a threshold risk. In this variation, in response to the time duration falling below a time threshold, the computer system can: generate the notification for the medical provider, the notification indicating the time duration and the corresponding risk and prompting the medical provider to investigate the user for prescription of a dose of an pharmacological medication; and serve the notification to the care provider.

In one example, in response to receiving the notification, the care provider may: view the series of physiological biosignal data, the series of motion data, and a series of communication data of the user who is not yet diagnosed with major depression disorder (e.g., but experiencing non clinically-significant depressive symptoms); based on these data, confirm the risk of depression onset within the target time window; and, based on the target time window falling below a threshold duration (e.g., a day), investigate the user for diagnosis with major depression disorder.

Therefore, the computer system can enable the care provider to: monitor the depressive symptoms of a user experiencing non-clinically significant depressive symptoms; predict the time duration until risk of depression relapse (or onset) by the user will exceed the threshold risk; and if the time duration is short—suggesting high risk of imminent onset of the set of depressive symptoms of the user—investigate the user for diagnosis with major depression disorder. In addition, the computer system can enable the care provider to prioritize arranging a medical appointment with the user who may begin to exhibit the set of depressive symptoms sooner than other users.

### 2.3 Other Diseases

Generally, Blocks of the method S100 can be executed by the computer system to calculate a risk of a user (e.g., patient) exhibiting a set of symptoms within a target time window, the symptoms attributable to of any complex multi-symptom disorder including clinical depression and generalized anxiety. In addition, Blocks of the method S100 can be executed to assess the risk based on a variety of user data including physiological data, motion data, communication data, self-evaluations, clinical evaluations, etc. Furthermore, Blocks of the method S100 can be executed to calculate a probability, likelihood, or other statistical measure indicating the likelihood of the user exhibiting the set of symptoms.

Furthermore, Blocks of the method S100 can be executed: to access a clinical assessment for depression of each user in a population of users; derive correlations between user data and this clinical assessment for depression; and compile these correlations into a depression model configured to predict risk of future depression diagnosis. However, Blocks of the method S100 are also applicable to: accessing a clinical assessment for anxiety and/or other complex multi-symptom disorder of each user in a population of users; deriving correlations between user data and this clinical assessment; and compiling the correlations into a model configured to predict risk of future anxiety disorder diagnosis and/or other complex multi-symptom disorder diagnosis.

In addition, Blocks of the method S100 can be executed to, prior to presentation of depression symptoms by a user, calculate: a risk of depression relapse (or onset) by a user within a threshold time window; and/or a time duration until risk of depression relapse (or onset) by a user will exceed a threshold risk. However, Blocks of the method S100 are also applicable to, prior to presentation of anxiety and/or other complex multi-symptom disorder symptoms by the user, calculating: a risk of developing anxiety and/or other complex multi-symptom disorder symptoms within a threshold time window; and/or a time duration until risk of developing the anxiety and/or other complex multi-symptom disorder will exceed a threshold risk.

### 3. Population Data Collection

Generally, Blocks S110, S112, and S114 of the method S100 recite: during a first time period, for each user in a population of users: accessing a clinical assessment for depression in the user; accessing a set of biosignal data (e.g., heart-rate, heart-rate variability, skin temperature, skin moisture, electrodermal activity, etc.) of the user, preceding the clinical assessment for depression; and accessing a first set of motion data (e.g., data collected by the mobile device of the user, geolocation data, acceleration data, Bluetooth data) of the user, preceding the clinical assessment for depression. Generally, in Blocks S110, S112, and S114, the computer system can access biosignal and motion data of each user in the population of users (e.g., population of users diagnosed with depression), the data indicative of mental state of each user in the population of users. In addition, the computer system can access the clinical assessment for depression of each user in the population of users, the clinical assessment for depression provided by the care provider (e.g., psychologist, therapist, coach) of each user in the population of users.

In one implementation, the computer system can access the clinical assessment for depression of each user in the population of users by: prompting the care provider to recount the clinical appearance, emotional state, and mental state of the user; and receiving a textual description of the clinical appearance, emotional state, and mental state of the user from the care provider. For example, the computer system can prompt the care provider to recount the mental state of the user following an appointment between the user in the population of users and the care provider. In particular, the computer system can: provide the care provider with a text input box via a user interface of a device of the care provider; and receive the textual description of the mental state of the user via the text input box. Additionally, or alternatively, the computer system can prompt the care provider to rate the state (e.g., mental state, emotional awareness, appearance, compliance) of the first user on a rating scale following the appointment. In particular, the computer system can access the clinical assessment for depression for each user in the population of users by: prompting the care provider to provide a set of scores (e.g., metrics, questionnaire responses, quantitative measures) representing the mental state of the user; and receive the set of scores (representing the mental state of the user from the care provider.

In one implementation, in addition to accessing the set of biosignal data, the second set of motion data, and the clinical assessment for depression of each user in the population of users, the computer system can access a set of communication data (e.g., voice, video, text, user behavioral data) from the mobile device of the user, the communications data indicative of social activity level of the user, which can be correlated with depression. Additionally, or alternatively, the computer system can access voice communications generated by each user in the population of users; and extract a first set of language signals from the set of text communications.

### 4. Population Marker Generation

Generally, Block S116 of the method S100 recites: transforming the set of biosignal data into a set of psychophysiological markers, the set of psychophysiological markers representing metrics associated with mental health of the user in the population of users. Generally, in Block S116, the computer system can transform the set of biosignal data, such as heart rate variability data, skin temperature data, and/or respiratory rate data, into the set of psychophysiological markers representing various metrics indicative of the mental health of the user, such as instances of target emotion (e.g., sadness), sleep quality, social activity level, psychomotor activity level.

In one variation, the computer system can: transform the set of biosignal data into a set of psychophysiological markers, the set of psychophysiological markers representing emotions exhibited by the user in the population of users.

In one implementation, the computer system can transform the set of biosignal data into the set of psychophysiological markers including sleep quality, periods of fatigue, and anxiety indicators exhibited by the user in the population of users.

Additionally, or alternatively, the computer system can transform the set of biosignal data, the set of motion data, the set of communication data, the set of language signals, and/or a set of other data into the set of psychophysiological markers of a user in the population of users. Therefore, the computer system can transform various types of data of a user in the population of users into a set of psychophysiological markers. Alternatively, the computer system can forgo transforming the set of biosignal data, the set of motion data, the set of language signals, and/or other data into the set of psychophysiological markers of a user in the population of users.

In one implementation, the computer system can utilize a model (e.g., sleep model, mental stress model, fatigue model) to transform the set of biosignal data, representative of the psychomotor activity, body energy, sleep duration and quality, respiratory rate, and/or autonomic nervous system responsiveness, into the set of psychophysiological markers representing sleep quality, periods of fatigue, and anxiety indicators exhibited by the user in the population of users. In particular, the computer system can implement methods and techniques described below and in U.S. patent application Ser. Nos. 16/460,105 and 18/126,100, each of which is incorporated herein by reference, to construct individualized models for transforming biosignal data into emotions for each user in this user population. Therefore, the computer system can transform the biosignal data of each user in the population of users into a set of psychophysiological markers indicative of the emotional, mental, and physiological health of each user in the population of users.

### 5. Depression Model Generation

Generally, Block S118 of the method S100 recites deriving a set of correlations between: the set of psychophysiological markers and the clinical assessment for depression; and the first set of motion data and the clinical assessment for depression. Block S120 of the method S100 recites compiling sets of correlations, derived for the population of users, into a depression model configured to predict risk of future depression diagnosis based on historical psychophysiological markers and historical motion data. Generally, in Blocks S118 and S120, the computer system can compile sets of correlations between clinical assessments for depression and sets of psychophysiological markers/sets of motion data, derived for the population of users, into the depression model configured to predict risk of depression diagnosis (e.g., relapse or onset) of a user. In one variation, the computer system can derive the set of correlations between: the set of biosignal data and the clinical assessment for depression; and the set of motion data and the clinical assessment for depression.

Accordingly, the computer system can identify patterns between the clinical assessments for depression of a user in the population of users and data (e.g., the set of biosignal data, the set of motion data, the set of psychophysiological markers, the set of language signals) of the user. Based on these patterns, the computer system can predict clinical assessments for a new user (e.g., user not in the population of users) from the data (e.g., biosignal data, motion data, psychophysiological markers, language signals) of the new user.

In one implementation, the computer system can access the clinical assessment of a user in the population of users by: prompting the care provider to recount mental state of the user (e.g., following an appointment between the user and the care provider); receive a textual description of mental state of the user from the care provider; and extract a second set of language signals from the textual description of the mental state of the user. In one example, the set of language signals can include a signal embedding (e.g., vector) representing the meaning of the textual description. In this implementation, the computer system can derive the set of correlations between the first set of psychophysiological markers and the clinical assessment for depression by deriving the set of correlations between the set of psychophysiological markers and the second set of language signals. In the example, the computer system can: transform the set of psychophysiological markers into a second embedding representing depression symptoms of the user; and utilize a transformer deep neural network architecture to derive the set of correlations between the embedding and the second embedding.

In one implementation, during the first time period, the computer system can: access a set of text communications generated by each user in the population of users; and extract a set of language signals from the set of text communications. In this implementation, the computer system can further derive the set of correlations between the set of language signals and the clinical assessment for depression. Accordingly, the computer system can identify patterns between the clinical assessment for depression of the user in the population of users and text data (or voice data), representative of the social activity level of the user, collected from the mobile device of the user in the population of users. Based on these patterns, the computer system can predict a clinical assessment for depression of a new user (e.g., a user not in the population of users) from a set of text communications obtained from a mobile device of the new user.

### 6. Depression Model Refinement

In one implementation, during the first time period, the computer system can, for each user in the population of users: access a set of self-assessments of depression symptoms generated by the user; and extract a series of depression symptom severities from the set of self-assessments. For example, the computer system can access questionnaire responses (e.g., PHQ-9 responses, GAD-7 responses, etc.) that reflect depressive symptom and anxiety symptom severity of each user in the population of users.

Then, the computer system can derive the set of correlations further between: the set of psychophysiological markers and the series of depression symptom severities; and the first set of motion data and the series of depression symptom severities. Additionally, or alternatively, the computer system can derive the set of correlations further between: the set of biosignal data and the series of depression symptom severities; the first set of motion data and the series of depression symptom severities; and/or the set of language signals and the series of depression symptom severities. Generally, the computer system can derive the set of correlations between the depression symptom severities and various types of user data, such as the set psychophysiological markers, the set of motion data, the set of biosignal data, and/or the set of language signals. Accordingly, the computer system can identify patterns between the series of depression symptom severities, obtained from self-assessments, of a user in the population of users and data of the user such as the set psychophysiological markers, the set of motion data, the set of biosignal data, the set of language signals. Based on these patterns, the computer system can predict changes in symptom severity of the user in the population of users from text data, biosignal data, and/or motion data.

In this implementation, the computer system can fine-tune the depression model to predict risk of future depression diagnosis and future depression symptom severity based on historical psychophysiological markers and historical motion data. In particular, the computer system can incorporate the set of correlations (e.g., correlations between the series of depression symptom severities and the set of psychophysiological markers, the first set of motion data, and/or the set of biosignal data) into the depression model.

In one implementation, the computer system can: access a first subset of biosignals of a user; convert the first subset of biosignals of the user into a first emotion; in response to the first emotion of the user comprising a target emotion, prompt the user to supply a first current personal depression symptom severity; and store the first current personal depression symptom severity in a first self-assessment in the set of self-assessments. Then, the computer system can: extract a first depression symptom severity, in the series of depression symptom severities, from the first self-assessment. Therefore, the computer system can prompt the user in the population of users to respond to a questionnaire (e.g., PHQ-9) of depression symptom severity in response to detecting the first emotion (e.g., sadness) based on the subset of biosignal.

In one example, the computer system can prompt the user to respond to a set of prompts by rating depression symptom severity on a rating scale and receiving the self-assessment from the user, the self-assessment including a set of ratings of depression symptom severity according to the rating scale. Then, the computer system can extract a depression symptom severity score in the series of depression symptom severities from the self-assessment in the set of self-assessments by calculating the depression symptom severity score, in the set of depression symptom severities, based on the set of ratings of depression symptom severity corresponding to the self-assessment. In one example, the computer system can add the set of ratings of depression system severity associated with the self-assessment to yield the depression symptom severity score.

### 7. Individual User Data Collection

Generally, Blocks S112 and S114 of the method S100 recite, during a second time period: accessing a series of biosignal data (e.g., heart-rate, heart-rate variability, skin temperature, skin moisture, electrodermal activity, etc.) collected by a wearable device worn by a first user; and accessing a series of motion data (e.g., data collected by the mobile device of the user, geolocation data, acceleration data, Bluetooth data) of the first user. Generally, in Blocks S112 and S114, the computer system can access biosignal and motion data of the first user, the first user either diagnosed with clinical depression or exhibiting subclinical depressive symptoms.

In one implementation, the computer system can assess the series of biosignal data collected by a set of sensors embedded in the wearable device worn by a first user, the series of biosignal data including electrodermal activity data, heart rate data, heart rate variability data; skin temperature data; and inertial measurement unit data.

In one implementation, the computer system can access the series of motion data of the first user from a mobile device of the user, the series of motion data including global positioning system data and accelerometer data indicative of the level of activity of the first user. In addition, the computer system can access communication data (e.g., voice, video, text, user behavioral data) from the mobile device of the first user, the communication data including voice and text communications of the first user indicative of the level of social activity of the user.

### 8. Marker Generation for Individual User

Generally, Block S116 of the method S100 recites: transforming the series of biosignal data into a series of psychophysiological markers. Generally, in Block S116, the computer system can transform the series of biosignal data, such as heart rate variability, skin temperature, respiratory rate, into the series of psychophysiological markers, such as instances of target emotion, level of fatigue, sleep quality.

In particular, the computer system can utilize a model (e.g., sleep model, mental stress model, fatigue model) to transform the series of biosignal data representative of the psychomotor activity, body energy, sleep duration and quality, respiratory rate, and autonomic nervous system responsiveness, into the series of psychophysiological markers representing instances of a target emotion (e.g., sadness, happiness, anger) of the first user measures of sleep quality of the user, measures of fatigue of the user, measures of anxiety of the user, etc. Therefore, the computer system can transform the biosignal data of the first user into series of psychophysiological markers indicative of the emotional, mental, and physiological health of the first user.

### 8.1 Example: Emotion Marker Interpretation

In one implementation, as described in U.S. patent application Ser. No. 16/460,105, during an initial time period preceding the second time period, the computer system can: prompt the first user to orally recite a story associated with a first target emotion (e.g., sadness); record a voice recording of the first user reciting the story; record an initial set of biosignal data via the wearable device worn by the first user; extract an initial set of psychophysiological markers from the voice recording, the initial set of psychophysiological markers including a first emotion marker for a first instance of the first target emotion exhibited by the first user during the first time period; label the initial set of biosignal data according to the initial set of psychophysiological markers to generate an emotion-labeled set of biosignal data; and generate a first emotion model linking biosignals to psychophysiological markers for the first user based on the emotion-labeled set of biosignal data. In particular, the computer system can: extract a series of pitch, voice speed, voice volume, tone, and/or other characteristics of voice of the first user from the voice recording; and transform this series into timestamped instances (and magnitudes) of the target emotion exhibited by the first user while reciting the story. The remote computer system can then: synchronize the series of biosignal data and series of instances of the target emotion; and implement machine learning, and/or other techniques to derive the first emotion model. Therefore, the computer system can generate the first emotion model configured to transform the series of biosignal data into the series of psychophysiological markers, the series of psychophysiological markers representing instances of the first target emotion (e.g., sadness) experienced by the first user. In this implementation, the computer system can transform the series of biosignal data into the series of psychophysiological markers based on the first emotion model.

### 8.2 Example: Health Indicator Interpretation

Additionally, or alternatively, as described in U.S. patent application Ser. No. 18/126,100, during the initial time period preceding the second time period, the computer system can: record an initial set of biosignal data via the wearable device worn by the first user; access an initial set of psychophysiological markers derived from a series of health evaluations (executed by the care provider or by the first user) representative of a set of health indicators for the first user; and generate a second emotion model linking biosignals to psychophysiological markers for the first user based on the initial set of biosignal data and the initial set of psychophysiological markers. In this implementation, the computer system can transform the series of biosignal data into the series of psychophysiological markers based on the second emotion model.

For example, the computer system can prompt the first user to execute a series of health evaluations configured to evaluate the set of health indicators (e.g., fatigue, energy level, mood) exhibited by the first user over a test period. Additionally, or alternatively, the computer system can prompt the care provider to execute a series of health evaluations (e.g., texts) configured to evaluate the set of health indicators (e.g., insomnia, cognitive performance) exhibited by the first user over a test period. During execution of these health evaluations, the wearable device can record the series of biosignal data of the first user via and offload the series of biosignal data to the computer system, such as in real-time or in intermittent data packets. The computer system can then: extract the set of health indicators (e.g., as a set of energy levels, a set of moods) exhibited by the first user during the test period based on results of the series of health evaluations; synchronize the set of health indicators with the series of biosignal data; and implement machine learning, deep learning, and/or other techniques to the second emotion model (e.g., fatigue model, mood model) linking the biosignals and the set of health indicators (e.g., energy levels, mood) exhibited by the first user. The computer system can then utilize the second emotion model to transform the series of biosignal data into the series of psychophysiological markers representing energy levels of the first user, mood of the first user, etc.

### 9. Risk Score Calculation

Generally, Blocks S130 and S32 of the method S100 recite: accessing a target time window; and, prior to presentation of a set of depression symptoms by the first user, calculating a first risk score representing risk of presentation of the set of depression symptoms by the first user within the target time window based on the series of psychophysiological markers, the series of motion data, and the depression model. Generally, in Blocks S130 and S32, the computer system can: set the target time window and, given the target time window, calculate the risk score of the first user developing depression symptoms within the target time window.

In one implementation, prior to presentation of the set of depression symptoms by the first user, the computer system can calculate the first risk score representing risk of presentation of depression symptoms by the first user within the target time window based on the first series of biosignal data, the series of motion data, the series of psychophysiological markers, the series of language signals, and/or any other series of data. Therefore, the computer system can calculate the first risk score based on various types of data collected from the first user.

In one implementation, the computer system can access the target time window by setting the target time window based on: historic responsiveness of the first user to the pharmacological medication (e.g., antidepressant medication); and anticipated effective period of the first dose of the pharmacological medication. In one example, the computer system can set a longer target time window for the first user with high historic responsiveness to the pharmacological medication than for a second user with low historic responsiveness to the pharmacological medication. In addition, the computer system can set a longer target time window for the first user prescribed the pharmacological medication with a longer effective period than for a second user prescribed a second pharmacological medication with a shorter effective period. Additionally, or alternatively, the computer system can set the target time window based on the responsiveness of the medical provider of the first user to receiving the notification.

In one implementation, the computer system can: access a threshold depression symptom severity; and, prior to presentation by the first user of depression symptom severity greater than the threshold depression symptom severity, calculate the first risk score representing presentation of the set of depression symptoms, approximating the threshold depression symptom severity, by the first user within the target time window. Therefore, the computer system can define the set of depression symptoms based on the threshold depression symptom severity (e.g., minimum severity of a set of depression symptoms attributable to depression) and calculate the first risk score representing presentation of the set of depression symptoms, approximating the threshold depression symptom severity.

### 10. Time Duration Calculation

In one variation, Blocks S130, S132, and S134 of the method S100 recite: accessing a risk threshold; and, prior to presentation of a set of depression symptoms by the first user, calculating a first time associated with a risk of presentation of the set of depression symptoms by the first user exceeding the risk threshold based on the series of psychophysiological markers and the depression model and calculating a first time duration between the first time and a current time. Generally, in Blocks S130, S132, and S134, the computer system can, select the risk threshold and, given the risk threshold, calculate a time duration for the risk of presentation of the set of depression symptoms by the first user exceeding the risk threshold.

In one implementation, the computer system can: access the threshold depression symptom severity; and prior to presentation by the first user of depression symptom severity, greater than the threshold depression symptom severity, calculate the first time duration associated with risk of presentation by the first user of the set of depression symptoms, approximating the threshold depression symptom severity, within the time duration. Therefore, the computer system can define the set of depression symptoms based on the threshold depression symptom severity (e.g., minimum severity of a set of depression symptoms attributable to depression) and calculate the first risk score representing presentation of the set of depression symptoms, approximating the threshold depression symptom severity.

In one implementation, the computer system can: access the risk threshold; and, prior to presentation by the first user of the set of depression symptoms, calculate the first time associated with the risk of presentation by the first user of the set of depression symptoms exceeding the risk threshold based on the series of psychophysiological markers, the series of biosignal data, the series of motion data, the first series of language signals, and/or a series of other data. Therefore, the computer system can calculate the first time duration to threshold risk based on various types of data collected from the first user.

In one implementation, the computer system can calculate the first time duration by calculating the first time associated with risk of presentation by the first user of the set of depression symptoms exceeding the relapse risk threshold, the set of depression symptoms: indiscernible to a nominal practicing physician during the first time duration; and visible to the nominal practicing physician after the first time.

### 11. Notification Generation

Generally, Blocks S140 and S150 of the method S100 recite: in response to the first risk score exceeding a threshold risk, populating the notification with the first risk score and a prompt to investigate the first user for prescription of an intervention (e.g., pharmacological medication, therapy); and serving the notification to a care provider associated with the first user. Generally, in Blocks S140 and S150, in response to the first risk score exceeding the threshold risk, the computer system can, present the care provider of the first user with the notification indicating the first risk score and a prompt to investigate the first user for prescription of the intervention (e.g., review the series of biosignal data of the first user, review the series of psychophysiological markers of the first user, review the set of self-evaluations of the first user, set up an appointment with the first user).

In one variation, Blocks S140 and S150 recite: in response to the first time duration (e.g., first time to the first time associated with risk of presentation of the set of depression symptoms by the first user, the risk exceeding the risk threshold) falling below a threshold duration, populating a notification with the first time duration; and a prompt to investigate the first user for prescription of a first dose of a pharmacological medication; and serving the notification to a care provider associated with the first user. In this variation, in Blocks S140 and S150, in response to the first time duration falling below the threshold time duration, the computer system can, present the care provider of the first user with the notification indicating the first time duration and a prompt to investigate the first user for prescription of a first dose of a pharmacological medication.

In one implementation, the computer system can further populate the notification with the first series psychophysiological markers and the series of motion data. Additionally, or alternatively, the computer system can further populate the notification with the first series of biomarkers, the first set of self-assessments, and/or the first series of language signals. Therefore, the computer system can populate the notification with the data of the first user collected during the second time period. The care provider may: review the data to confirm the first risk score; discuss this data with the first user during an appointment between the first user and the care provider; and utilize this data to inform a treatment plan for the first user.

In one implementation, in response to the first risk score exceeding a threshold risk (or in response to the first time duration falling below threshold duration), the computer system can: populate a second notification with the first risk score (or the first time duration) and the first series of biomarkers, the first set of self-assessments, and/or the first series of language signals; and serve the second notification to the first user. Therefore, in response to the first risk score exceeding a threshold risk (or in response to the first time duration falling below a threshold duration), the computer system can notify the first user of the first risk score and present the first user with the data used to generate the first risk score.

In one implementation, serving the notification to the care provider can include: encrypting the notification; and transmitting the notification through an encrypted electronic messaging channel. Similarly, the computer system can encrypt the second notification sent to the first user and transmit the second notification through the encrypted electronic messaging channel. Accordingly, the computer system can encrypt notifications transmitted to the care provider or the first user, thereby ensuring privacy of the first user and protecting the first risk score, an identity of the first user, and the data of the first user (e.g., first series of biosignal) from access by third parties.

### 11.1 Example: Prescription Change

In one implementation, the computer system can access a first medical record of the first user, the first medical record specifying: a chronic depression diagnosis of the first user; and a current dose of the pharmacological medication prescribed to the first user. Then, in response to the first time duration (e.g., first time duration to the first time associated with a risk of presentation of the set of depression symptoms by the first user, the risk exceeding the risk threshold) falling below a target minimum duration between doses specified for the pharmacological medication, the computer system can populate the notification with the prompt to investigate the first user for prescription of the first dose of pharmacological medication exceeding the current dose of the pharmacological medication. Therefore, the computer system can prompt the care provider to adjust the dose of the pharmacological medication prescribed to the first user diagnosed with chronic depression in response to the first time duration falling below a target minimum duration between doses specified for the pharmacological medication, which can indicate a possible worsening of depression symptoms of the first user within the first time duration.

### 11.2 Example: Prescription Renewal

In one implementation, the computer system can access a first medical record of the first user, the first medical record indicating: episodic depression of the first user; and a previous dose of the pharmacological medication prescribed to the first user. In this implementation, in response to the first risk score exceeding a threshold risk, the computer system can populate the notification with the prompt to investigate the first user for renewal of prescription of the previous dose of the pharmacological medication. Therefore, the computer system can prompt the care provider to renew the prescription of the dose of the pharmacological medication for the first user diagnosed with episodic depression in response to the first risk score exceeding a threshold risk, which can indicate a possible depression relapse or onset within the target time window.

### 11.3 Example: Depression Diagnosis

In one implementation, the computer system can access a first medical record of the first user, the first medical record specifying non-clinically significant depressive anxiety symptoms of the first user. In this implementation, in response to absence of a current clinical depression diagnosis in the first medical record, the computer system can populate the notification with a second prompt to investigate the first user for a clinical depression diagnosis. Therefore, computer system can prompt the care provider to investigate the first user (e.g., the first user not diagnosed with depression but exhibiting depressive symptoms) for diagnosis with depression in response to the time duration falling below the threshold duration, which can indicate a worsening of the depressive symptoms of the first user.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during a first time period:
for each user in a population of users:
accessing a clinical assessment for depression in the user;
accessing a first set of biosignal data, of the user, preceding the clinical assessment for depression;
accessing a first set of motion data, of the user, preceding the clinical assessment for depression;
transforming the first set of biosignal data into a first set of psychophysiological markers, the first set of psychophysiological markers comprising emotions exhibited by the user; and
deriving a set of correlations between:
the first set of psychophysiological markers and the clinical assessment for depression; and
the first set of motion data and the clinical assessment for depression; and
compiling sets of correlations, derived for the population of users, into a depression model configured to predict risk of future depression diagnosis based on historical psychophysiological markers and historical motion data; and
during a second time period:
accessing a first series of biosignal data collected by a wearable device worn by a first user;
accessing a first series of motion data of the first user;
transforming the first series of biosignal data into a first series of psychophysiological markers;
accessing a target time window;
prior to presentation of a set of depression symptoms by the first user, calculating a first risk score representing presentation of the set of depression symptoms by the first user within the target time window based on the first series of psychophysiological markers, the first series of motion data, and the depression model;
in response to the first risk score exceeding a threshold risk, populating a notification with:
the first risk score; and
a prompt to investigate the first user for prescription of a first dose of an pharmacological medication; and
serving the notification to a care provider associated with the first user.

2. The method of claim 1:
further comprising:
during the first time period:
for each user in the population of users:
accessing a set of self-assessments of depression symptoms generated by the user; and
extracting a series of depression symptom severities from the set of self-assessments;
wherein deriving the set of correlations comprises deriving the set of correlations further between:
the first set of psychophysiological markers and the series of depression symptom severities; and
the first set of motion data and the series of depression symptom severities;
wherein compiling sets of correlations, derived for the population of users, into the depression model comprises generating the depression model configured to predict risk of future depression diagnosis and future depression symptom severity based on historical psychophysiological markers and historical motion data;
further comprising accessing a threshold depression symptom severity; and
wherein calculating the first risk score comprises:
prior to presentation of depression symptom severity, greater than the threshold depression symptom severity, by the first user:
calculating the first risk score representing presentation of the set of depression symptoms, approximating the threshold depression symptom severity, by the first user within the target time window.

3. The method of claim 2:
wherein accessing a first set of biosignal data for each user in the population of users comprises:
accessing a first subset of biosignals of a user;
wherein transforming a first set of biosignal data into a first set of psychophysiological markers for each user in the population of users comprises:
converting the first subset of biosignals of the user into a first emotion;
wherein accessing the set of self-assessments for each user in the population of users comprises:
in response to the first emotion of the user comprising a target emotion:
prompting the user to supply a first current personal depression symptom severity; and
storing the first current personal depression symptom severity in a first self-assessment in the set of self-assessments; and
wherein extracting the series of depression symptom severities from the set of self-assessments comprises:
extracting a first depression symptom severity, in the series of depression symptom severities, from the first self-assessment.

4. The method of claim 1:
further comprising:
for each user in the population of users:
accessing a first set of text communications generated by the user; and
extracting a first set of language signals from the first set of text communications;
wherein deriving the set of correlations comprises deriving the set of correlations further between:
the first set of language signals and the clinical assessment for depression; and
further comprising, during the second time period:
accessing a first series of text communications generated by the first user; and
extracting a first series set of language signals from the first set of text communications; and
wherein calculating the first risk score comprises calculating the first risk score further based on the first series set of language signals.

5. The method of claim 1:
wherein accessing the clinical assessment comprises:
for each user in the population of users:
prompt the care provider to recount emotional state of the user;
receive a textual description of emotional state of the user from the care provider; and
extract a second set of language signals from the textual description of the emotional state of the user; and
wherein deriving the set of correlations between the first set of psychophysiological markers and the clinical assessment for depression comprises deriving the set of correlations between the first set of psychophysiological markers and the second set of language signals.

6. The method of claim 1:
further comprising, during an initial time period preceding the second time period:
prompting the first user to orally recite a story associated with a first target emotion;
recording a voice recording of the first user reciting the story;
recording an initial set of biosignal data via the wearable device worn by the first user;
extracting an initial set of psychophysiological markers from the voice recording, the initial set of psychophysiological markers comprising a first emotion marker for a first instance of the first target emotion exhibited by the first user during the first time period;
labeling the initial set of biosignal data according to the initial set of psychophysiological markers to generate an emotion-labeled set of biosignal data; and
generating a first emotion model linking biosignals to psychophysiological markers for the first user based on the emotion-labeled set of biosignal data; and
wherein transforming the first series of biosignal data into the first series of psychophysiological markers comprises:
transforming the first series of biosignal data into the first series of psychophysiological markers based on the first emotion model.

7. The method of claim 1:
further comprising, during an initial time period preceding the second time period:
recording an initial set of biosignal data via the wearable device worn by the first user;
accessing an initial set of psychophysiological markers derived from a series of health evaluations executed by the care provider for the first user and representative of a set of health indicators for the first user; and
generating a second emotion model linking biosignals to psychophysiological markers for the first user based on the initial set of biosignal data and the initial set of psychophysiological markers; and
wherein transforming the first series of biosignal data into the first series of psychophysiological markers comprises:
transforming the first series of biosignal data into the first series of psychophysiological markers based on the second emotion model.

8. The method of claim 1, wherein transforming the first set of biosignal data into the first set of psychophysiological markers comprises transforming the first set of biosignal data into the first set of psychophysiological markers further comprising sleep quality, periods of fatigue, and anxiety indicators exhibited by the user.

9. The method of claim 1, wherein populating the notification further comprises:
encrypting the first series of psychophysiological markers and the first series of motion data to generate an encrypted first series of psychophysiological markers and an encrypted first series of motion data; and
populating the notification with the encrypted first series of psychophysiological markers and the encrypted first series of motion data.

10. The method of claim 1:
further comprising accessing a first medical record of the first user, the first medical record indicating:
episodic depression of the first user; and
a previous dose of the pharmacological medication prescribed to the first user;
wherein populating the notification with the prompt comprises populating the notification with the prompt to investigate the first user for renewal of prescription of the previous dose of the pharmacological medication; and
further comprising:
populating a second notification with the first risk score and indicating renewed prescription of the previous dose of the pharmacological medication; and
serving the second notification to the first user.

11. The method of claim 1, wherein serving the notification comprises:
encrypting the notification; and
transmitting the notification through an encrypted electronic messaging channel.

12. The method of claim 1, wherein accessing the target time window comprises:
setting the target time window based on:
historic responsiveness of the first user to the pharmacological medication; and
anticipated effective period of the first dose of the pharmacological medication.

13. A method comprising:
during a first time period:
for each user in a population of users:
accessing a clinical assessment for depression in the user;
accessing a first set of biosignal data, of the user, preceding the clinical assessment for depression;
accessing a first set of motion data, of the user, preceding the clinical assessment for depression;
transforming the first set of biosignal data and the first set of motion data into a first set of psychophysiological markers of the user;
deriving a set of correlations between:
the first set of psychophysiological markers and the clinical assessment for depression; and
compiling sets of correlations, derived for the population of users, into a depression model configured to predict risk of future depression diagnosis based on historical psychophysiological markers; and
during a second time period:
accessing a first series of biosignal data collected by a wearable device worn by a first user;
accessing a first series of motion data of the first user;
transforming the first series of biosignal data and the first series of motion data into a first series of psychophysiological markers;
accessing a risk threshold;
prior to presentation of a set of depression symptoms by the first user:
calculating a first time associated with a risk of presentation of the set of depression symptoms by the first user based on the first series of psychophysiological markers and the depression model, the risk exceeding the risk threshold; and
calculating a first time duration between the first time and a current time; and
in response to the first time duration falling below a threshold duration:
populating a notification with:
the first time duration; and
a prompt to investigate the first user for prescription of a first dose of an pharmacological medication; and
serving the notification to a care provider associated with the first user.

14. The method of claim 13:
further comprising:
during the first time period:
for each user in the population of users:
accessing a set of self-assessments of depression symptoms generated by the user; and
extracting a series of depression symptom severities from the set of self-assessments;
wherein deriving the set of correlations comprises deriving the set of correlations further between the first set of psychophysiological markers and the series of depression symptom severities;
wherein compiling sets of correlations, derived for the population of users, into the depression model comprises generating the depression model configured to predict risk of future depression diagnosis and future depression symptom severity based on set of psychophysiological markers;
further comprising accessing a threshold depression symptom severity; and
wherein calculating the first time duration comprises:
prior to presentation of depression symptom severity, greater than the threshold depression symptom severity, by the first user:
calculating the first time duration associated with risk of presentation of the set of depression symptoms, approximating the threshold depression symptom severity, by the first user within the first time duration.

15. The method of claim 13:
further comprising, during an initial time period preceding the second time period:
by a computing device carried by the first user:
prompting the first user to orally recite a story associated with a first target emotion;
recording a voice recording of the first user reciting the story; and
recording an initial set of biosignal data via the wearable device worn by the first user;
extracting an initial set of psychophysiological markers from the voice recording, the initial set of psychophysiological markers comprising a first emotion marker for a first instance of the first target emotion exhibited by the first user during the first time period;
labeling the initial set of biosignal data according to the initial set of psychophysiological markers to generate an emotion-labeled set of biosignal data; and
generating an emotion model linking biosignals to psychophysiological markers for the first user based on the emotion-labeled set of biosignal data; and
wherein transforming the first series of biosignal data into the first series of psychophysiological markers comprises transforming the first series of biosignal data into the series of psychophysiological markers based on the emotion model.

16. The method of claim 13:
further comprising:
accessing a first medical record of the first user, the first medical record specifying non-clinically significant depressive anxiety symptoms of the first user; and
wherein populating the notification comprises:
in response to absence of a current clinical depression diagnosis in the first medical record:
populating the notification with a second prompt to investigate the first user for a clinical depression diagnosis.

17. The method of claim 13:
further comprising accessing a first medical record of the first user, the first medical record specifying:
a chronic depression diagnosis of the first user; and
a current dose of the pharmacological medication prescribed to the first user; and
wherein populating the notification comprises:
in response to the first time duration falling below a target minimum duration between doses specified for the pharmacological medication:
populating the notification with the prompt to investigate the first user for prescription of the first dose of pharmacological medication exceeding the current dose of the pharmacological medication.

18. The method of claim 13, wherein calculating the first time duration comprises calculating the first time associated with the risk of presentation of the set of depression symptoms by the first user exceeding the risk threshold, the set of depression symptoms:
indiscernible to a nominal practicing physician during the first time duration; and
visible to the nominal practicing physician after the first time.

19. A method comprising:
during a first time period:
for each user in a population of users:
accessing a clinical assessment for depression in the user;
accessing a first set of biosignal data, of the user, preceding the clinical assessment for depression;
accessing a first set of motion data, of the user, preceding the clinical assessment for depression;
deriving a set of correlations between:
the first set of biosignal data and the clinical assessment for depression; and
the first set of motion data and the clinical assessment for depression; and
compiling sets of correlations, derived for the population of users, into a depression model configured to predict risk of future depression diagnosis based on historical biosignal data and historical motion data;
during a second time period:
accessing a first series of biosignal data collected by a wearable device worn by a first user; and
accessing a first series of motion data collected by a mobile device of the first user;
accessing a target time window;
prior to presentation of a set of depression symptoms by the first user:
calculating a first risk score representing presentation of the set of depression symptoms by the first user within the target time window based on the first series of biosignal data and the first series of motion data; and
in response to the first risk score exceeding a threshold risk:
populating a notification with:
the first risk score; and
a prompt to investigate the first user for prescription of an intervention; and
serving the notification to a care provider associated with the first user.

20. The method of claim 19:
further comprising:
during the first time period:
for each user in the population of users:
accessing a set of self-assessments of depression symptoms generated by the user; and
extracting a series of depression symptom severities from the set of self-assessments;
wherein deriving the set of correlations comprises deriving the set of correlations further between:
the first set of biosignal data, the first set of motion data and the series of depression symptom severities; and
wherein compiling sets of correlations, derived for the population of users, into the depression model comprises generating the depression model configured to predict risk of future depression diagnosis and future depression symptom severity based on historical biosignal data and historical motion data.
